(19) United States
(12) Patent Application Publication
(10) Pub. No.: US 2005/0154033 A1
(43) Pub. Date: Jul. 14, 2005

(54) Methods of using zonisamide as an adjunctive therapy for partial seizures

(75) Inventor: Ivan Lieberburg, Berkeley, CA (US)

Correspondence Address: Finnegan, Henderson, Farabow,;Garrett & Dunner, L.L.P., 1300 I Street, N.W., Washington, DC, 20005-3315, US

(73) Assignee: Eisai Co., Ltd, Tokyo (JP)

(21) Appl. No.: 10/752,522

(22) Filed: Jan. 8, 2004

(51) Int. Cl. A61K 31/42

(52) U.S. Cl. 514/379

(57) ABSTRACT

Methods of using zonisamide as an adjunctive therapy for partial seizures are disclosed. In particular, the methods enhance the safety of patients taking pharmaceutical formulations of zonisamide by providing information that increases the awareness of neuroleptic malignant syndrome (NMS) as a possible side effect; wherein the patients and/or prescribing physicians and other medical care providers are advised to monitor for NMS and employ methods that will improve the therapeutic outcome in the few patients who experience NMS associated with zonisamide therapy.

### FIELD OF THE INVENTION

The present invention generally relates to methods of using zonisamide (3-benzisoxazole methylene sulfonamide) as an adjunctive therapy for partial seizures.

### BACKGROUND OF THE INVENTION

In the United States, over 2 million serious adverse drug reactions (ADRs) occur ever year, with 100,000 associated deaths. This places ADRs as the fourth leading cause of death, ranking ahead of pulmonary disease, diabetes, AIDS, pneumonia, accidents, and automobile deaths. Compounding this problem is the fact that ADRs increase exponentially in patients who take four or more medications concurrently. (See http://www.fda.gov/cder/drug/drugReactions/default.htm, last checked Oct. 20, 2003.)

Most drugs are approved by a Food and Drug Administration review process after an average of 1,500 patient exposures. Clinical trials involving this number of subjects (both healthy volunteers and patients in need of the therapeutic effect of the drug under review) provide a statistically relevant sample of the population from which an assessment of safety and efficacy can be evaluated. However, some drugs have very rare toxicity profiles. Bromfenac, for example, causes hepatotoxicity in 1 out of 20,000 patients. For drugs with rare toxicity, more than 100,000 patients must be exposed to generate a warning signal for the adverse event. In instances where an adverse event is identified in association with a human therapeutic, government regulations require a post-approval follow-up after the drug has been taken to market.

Examples of very serious post-marketing events that have been identified in the recent past include Fen-Phen (fenfluramine—phentermine combination therapy) for weight loss and Rezulin (troglitazone) for diabetes, both of which were later removed from the market because the ADR risks outweighed the therapeutic benefits. Statistical and clinical analysis of large adverse event databases collected by post-marketing surveillance is one method by which identification of the rarer ADRs can be made. For more background on the occurrence and identification of ADRs see, for example, Lazarou, J. et al. JAMA 279(15):1200-1205 (1998), and Gurwitz, J. H. et al. Am J. Med. 109(2):87-94 (2000). For a discussion of techniques and difficulties inherent in identifying ADRs in adjunctive therapies of epileptic seizures, see French, J. Epilepsia 43(9): 951-955 (2002), which is hereby incorporated by reference in its entirety.

While Rezulin and Fen-Phen are notable for their extreme and potentially irreversible nature, other adverse drug reactions can be minimized or more easily reversed if they are recognized early, and appropriate and timely medical intervention is made. A few examples of frequently reversible adverse events are cardiac arrhythmias, liver function abnormalities, and irregularities in hematopoiesis. Thus, there remains a need for methods for identifying, for detecting and for treating adverse events associated with drug therapy, in a timely and informed manner.

### DESCRIPTION OF THE INVENTION

Unexpectedly it has been found by the applicants that zonisamide therapy in a very small percentage of patients (available estimates in the U.S. and Japan are about 1:37,276 and about 1:592,588, respectively) can precipitate neuroleptic malignant syndrome (NMS). It also has been found that by curtailing (either by removal, reduction, or tapering off) the administration of zonisamide dosing, alone or in conjunction with other concomitant medications, alleviation and minimization of this severe adverse event is possible. This is particularly the case when medical intervention to manage the disease and/or removal, reduction, or tapering off of zonisamide is instituted rapidly.

Accordingly, the present invention is directed to methods of using zonisamide for a regulatory agency approved use (e.g., as an adjunctive therapy for partial seizures). The methods improve the safety of zonisamide therapy for patients receiving administrations of the drug, or those who are in need of zonisamide therapy.

In some embodiments, the methods of using zonisamide as an adjunctive therapy for partial seizures improves the safety and health of patients taking zonisamide by increasing the awareness of the patient or patient's guardian that neuroleptic malignant syndrome (NMS) is a possible side effect. Accordingly, a patient may be provided with a therapeutically effective amount of zonisamide, and the patient or the patient's guardian may be informed that dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis are symptoms of NMS that require prompt medical evaluation if such symptoms are experienced by the patient. As a result, the patient or patient's guardian can monitor for signs and symptoms of NMS, and seek medical attention if such symptoms occur in order to obtain appropriate tests, diagnosis, and treatment. In some embodiments, the present methods reduce the risk of NMS in patients receiving zonisamide therapy.

In other embodiments, the present invention provides methods of using zonisamide as an adjunctive therapy for partial seizures comprising informing a prescribing physician or other medical professional (e.g., an emergency medical worker) that NMS may result from zonisamide therapy and to monitor a patient who is prescribed zonisamide as an adjunctive therapy for partial seizures for dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis. The prescribing physician or other medical professional also may be advised that when dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis is observed, an appropriate diagnostic be employed to determine whether NMS is present. In addition, the prescribing physician or other medical professional may be advised to remove, reduce, or taper off the zonisamide dosing in the patient, and initiate appropriate supportive therapy for the underlying condition(s). In this manner, the present methods enable prescribing physicians and other health care professionals to recognize and minimize the risk associated with an adverse event, namely NMS, which may occur in some patients who receive zonisamide therapy.

The present methods also include methods of administrating zonisamide as an adjunctive therapy for partial seizures comprising providing packaging that includes a pharmaceutical formulation of zonisamide along with information providing a warning that zonisamide may cause NMS in some patients and that one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis are potentially signs of NMS; and providing the packaging to a patient who has been prescribed zonisamide.

The present methods also include methods of administrating zonisamide as an adjunctive therapy for partial seizures to patients who are restrained, dehydrated, or have Parkinson's disease. The methods may improve the health of the patients, improve the safety of the zonisamide therapy for such patients, or decrease the risk of NMS. The methods comprise informing a prescribing physician or other medical professional (e.g., an emergency medical worker) that, in a patient that is restrained, dehydrated, or has Parkinson's disease, NMS may result from zonisamide therapy; and advising the physician or worker to monitor such a patient for signs and symptoms of NMS. The prescribing physician or other medical professional also may be advised that when signs and symptoms of NMS are observed in such patients, an appropriate diagnostic be employed to determine whether NMS is present. In addition, the prescribing physician or other medical professional may be advised to remove, reduce, or taper off the zonisamide dosing in the patient, and initiate appropriate supportive therapy for the underlying condition(s). In this manner, the present methods enable prescribing physicians and other health care professionals to recognize and minimize the risk associated with an adverse event, namely NMS, which may occur in some patients who are restrained, dehydrated, or have Parkinson's disease. The information described above may be provided as part of the packaging that is included with a pharmaceutical formulation of zonisamide, such as a package drug insert.

In some embodiments, the methods of using zonisamide as an adjunctive therapy for partial seizures improves the safety and health of patients taking zonisamide who are restrained, dehydrated, or have Parkinson's disease by increasing the awareness of the patient or patient's guardian that NMS is a possible side effect. Accordingly, a patient may be provided with a therapeutically effective amount of zonisamide, and the patient or the patient's guardian may be informed of the signs and symptoms of NMS that require prompt medical evaluation if such symptoms are experienced by the patient, as described above. As a result, the patient or patient's guardian can monitor for signs and symptoms of NMS, and seek medical attention if such symptoms occur in order to obtain appropriate tests, diagnosis, and treatment. In some embodiments, the present methods improve the safety of the zonisamide therapy, improve the health of the patient, or reduce the risk of NMS in patients receiving zonisamide therapy that are dehydrated, restrained, or have Parkinson's disease. The information described above may be provided as part of the packaging that is included with a pharmaceutical formulation of zonisamide, such as a package drug insert.

In cases where patients have Parkinson's disease and are taking zonisamide, the patient, patient's guardian, prescribing physician, or other medical professional may be advised to observe for signs and symptoms of NMS (as described above), in particular, after discontinuing zonisamide therapy. In such patients, the physician or other medical professional may be advised to institute appropriate treatment for NMS in Parkinson's disease. The information described above may be provided as part of the packaging that is included with a pharmaceutical formulation of zonisamide, such as a package drug insert.

The medical information provided in any of the above described methods concerning the signs and symptoms of NMS may alternatively be provided in layman's terms, so as to be better understood by patients or non-medical professionals. Those of skill in the medical art are familiar with the various layman's terms that can be used to describe the symptoms of NMS.

Other advantages and uses of the present invention will become apparent to those skilled in the art in studying this disclosure; therefore this recitation is not intended to limit the scope of the claims attached hereto.

### DESCRIPTION OF THE EMBODIMENTS

Zonisamide is an antiseizure drug, chemically classified as a sulfonamide and unrelated to other antiseizure agents. Antiepileptic drugs are commonly abbreviated as “AEDs.” The active ingredient is zonisamide, 1,2-benzisoxazole-3-methanesulfonamide. Zonisamide was approved in 2000 for the adjunctive treatment, i.e., taken in conjunction with one or more other AED, treatment of epilepsy in the United States. It was first introduced in Japan approximately 12 years ago, where it also has been used as monotherapy, i.e., without other AEDs as concomitant therapeutics. Zonisamide is not known to be a hepatic enzyme inducer and has been administered adjunctively with almost all of the other regulatory-approved AEDs either in the United States or abroad.

The precise mechanism(s) by which zonisamide exerts its anti-seizure effect is unknown. Zonisamide may produce antiseizure effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization, thus suppressing hyperexcitablity in epileptic foci. In vitro binding studies have demonstrated that zonisamide binds to the GABA/benzodiazepine receptor ionophore complex in an allosteric fashion, which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has weak carbonic anhydrase inhibiting activity (about 1/50^{th} the inhibition compared to acetazolamide), and this pharmacologic effect is not thought to be a major contributing factor in the anti-seizure activity of zonisamide.

ZONEGRAN® (the human therapeutic pharmaceutical formulation containing zonisamide) is indicated as adjunctive therapy for the treatment of partial seizures in adults and is supplied by prescription in the form of 25, 50, and 100 mg capsules. The capsule may be divided, so as to offer smaller increments in dosage. Recommended dosing is once or twice daily, the recommended daily dose of 100 mg at the initiation of therapy should not be divided. ZONEGRAN® is given orally and can be taken with or without food. While other therapeutic uses of zonisamide have been reported, such as treatment of obesity and eating disorders, treatment of neuropathic pain, prophylaxis of migraine attacks, and treatment of mania, these are not indications approved by the Food and Drug Administration (FDA) in the United States, and so are called “off-label” uses. Off-label uses, which are within the discretion of the prescribing physician to write, are also encompassed in the methods presented herein.

Prescribing physicians are informed in the product insert (which. contains prescribing information approved by the FDA) that, because of the long half-life of zonisamide, up to two weeks may be required to achieve steady-state levels upon reaching a stable dose or following dosage adjustment. Although the regimen described below has been shown to be tolerated, the prescriber may wish to prolong the duration of treatment at the lower doses in order to fully assess the effects of zonisamide at steady state, noting that many of the side effects of zonisamide are more frequent at doses of 300 mg per day and above. Although there is some evidence of greater response at doses above 100-200 mg/day, the increase appears small and formal dose-response studies have not been conducted.

The initial dose should be 100 mg daily. After two weeks, the dose may be increased to 200 mg/day for at least two weeks. It can be increased to 300 mg/day and 400 mg/day, with the dose stable for at least two weeks to achieve steady state at each level. Evidence from controlled trials suggests that ZONEGRAN® doses of 100-600 mg/day are effective, but there is no suggestion of increasing response above 400 mg/day.

Adjunctive therapy for partial seizures in adults denotes that these patients are already on other anti-epileptic medications, but that they are continuing to seize at a rate that has been deemed by their treating physician to require additional (add-on) therapy. For a recent review of AEDs currently available to American physicians, their efficacies for particular types of epileptic seizures and associated ADRs, see: Ilo Leppik, Epilepsia 42(Suppl.4): 1-6 (2001).

The use of multiple anti-epileptic medications in the adjunctive setting and other drug combinations increases the likelihood of confluent or interactive ADRs, and also may confuse the treating physician as to the causal agent. For instance, when an attending medical professional is presented with a patient taking a combination of medications and manifesting a particular side-effect, it is difficult to diagnose which of the patient's medications (or combination of medications) is responsible for the observed side effect. Typically, the attending physician must consult the medical literature of known adverse events to identify drug(s) that are most likely to cause the observed side-effects. Known adverse events may also be found in the package drug inserts for each drug. The drug(s) having the higher likelihood of causing the observed side-effects are usually reduced or withdrawn first. When such options are exhausted, the patient may have to be systematically withdrawn from the various drugs until the cause is identified. Since zonisamide is typically prescribed as an adjunctive therapy, it presents such complications when side-effects occur.

This situation is further complicated when side-effects occur that are not normally associated with a particular drug. For example, zonisamide was not previously known to be linked with NMS in patients receiving ZONEGRAN® therapy. However, antipsychotic drugs are known to cause NMS. Given this knowledge, a medical professional would not suspect zonisamide to be the likely agent responsible for causing NMS in a patient taking zonisamide alone, or in combination with antipsychotic drugs. In the case of a NMS-manifesting patient receiving a combination of zonisamide and antipsychotics, the patient would be withdrawn from the known causative agents (i.e., the antipsychotics). Meanwhile, the attending medical professional would have no obvious reason to withdraw such a patient from zonisamide, and would allow the therapy to continue while searching for the cause of the NMS.

However, a careful review of the data generated in American clinical trials, as well as in ADR reports gathered once commercial marketing began, has yielded the discovery that zonisamide may independently induce NMS in a small number of patients, and has implicated NMS in patients receiving zonisamide as an adjunctive therapy. Accordingly, the present invention is directed to methods of increasing the safety of zonisamide therapy in view of its newly discovered role in NMS.

Neuroleptic malignant syndrome (NMS) is a rare, but potentially fatal disease that often results from a reaction to a neuroleptic medication. For a general review of the disease and related literature, see Adnet, et al., “Neuroleptic Malignant Syndrome,” Br. J. Anaesthesia, Vol. 85, pp. 129-135, 2000; Caroff, et al., “Neuroleptic Malignant Syndrome,” Adverse Drug Bulletin, No. 209, pp. 799-802, August 2001; and Pelonero, et al., “Neuroleptic Malignant Syndrome: A Review,” Psychiatric Services, Vol. 49, pp.1163-1172, 1998. The overall incidence is uncommon, with rates ranging from 0.02 to 12.2% of patients being treated with a neuroleptic medication and about 0.07 to 0.2% overall. The syndrome is commonly characterized by fever, muscular rigidity, altered mental status, and autonomic dysfunction.

Known risk factors for developing NMS include dehydration; rapid initiation or dose escalation of some neuroleptic drugs; withdrawal of some anti-Parkinson medications; prior history of NMS; and use of predisposing drugs such as antipsychotics. While some clear risk factors for NMS are known, however, the low incidence of this syndrome and the consequent difficulty in studying it in a controlled, prospective manner make clinical features, predisposing conditions, treatment, and prognosis difficult to define.

Neuroleptics (dopamine D2-receptor antagonists) are associated with NMS. Although neuroleptic drugs such as haloperidol and fluphenazine are more frequently associated with NMS, all antipsychotic agents may cause the syndrome. Such agents include prochlorperazine (Compazine), promethazine (Phenergan), clozapine (Clozaril), and risperidone (Risperdal). NMS has also been associated with a few non-neuroleptic agents that block central dopamine pathways, e.g., metoclopramide (Reglan), amoxapine (Ascendin), and lithium. NMS has also been observed following the withdrawal of anti-Parkinson medications.

The dopamine receptor blockade, resulting in decreased dopamine activity in the central nervous system (CNS), is considered to be the primary cause of NMS. This may result either from a blockade of dopamine D2-receptors or a decreased availability of dopamine itself. For example, blockade of dopamine neurotransmission in the nigrostriatum and hypothalamus results in muscular rigidity and altered thermoregulation, respectively. Experimental blockade of dopamine in the striatum can cause rigidity, tremor, and rhabdomyolysis. Thus, there is evidence that such sympathetic nervous system activation or dysfunction may play a significant role in the pathogenesis of NMS.

The incidence of mortality from NMS is estimated at 5-11.6%. Fatalities typically result from respiratory failure, cardiovascular collapse, myoglobinuric renal failure, arrhythmias, or diffuse intravascular coagulation (DIC). Complications associated with NMS include rhabdomyolysis, pneumonia, renal failure, seizures, arrhythmias, DIC, and respiratory failure.

As an additional complication, patients with Parkinson's disease (PD) are at a higher risk for developing NMS. In patients with PD, an NMS-like condition has been reported following the withdrawal of anti-PD medications. However, the disorder also develops in patients that have not withdrawn from any anti-PD medications; and not all patients who are withdrawn from such drugs develop NMS. This suggests that some PD patients in the population are more susceptible than others to NMS. See Ueda, et al., “Susceptibility to Neuroleptic Malignant Syndrom in Parkinson's Disease,” Neurology, Vol. 52, pp. 777-781, 1999.

In order to protect these NMS-susceptible patients from the life-threatening effects of NMS, it is important to identify other factors that may be implicated in the development of the condition. Similarly, patients who have a history of NMS, are dehydrated or restrained may be at an increased risk of NMS. One of skill in the art is familiar with the various reports implicating risk factors that may render a patient more susceptible to the development of NMS, some of which are described above. In some embodiments, the present methods are directed to patients that are taking zonisamide and are at an increased risk for developing NMS, such as those patients that are dehydrated, restrained, or have Parkinson's disease. However, the present methods may also be used with patients taking zonisamide that meet the criteria for other known risk factors that increase the likelihood that the patient will develop NMS.

Diagnosis of NMS traditionally made on the basis of clinical history and manifestation of particular symptoms. Relevant clinical history includes prior initiation or dose escalation of neuroleptics (e.g., within the past 1-4 weeks); withdrawal of anti-Parkinson medications; use of predisposing drugs such as antipsychotics; and prior history of NMS. Symptoms of NMS include dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis.

Several laboratory tests are available to aid the diagnosis of NMS. Such tests include: complete blood count (CBC); blood cultures; liver function tests (LFTs); blood urea nitrogen (BUN) and creatinine; calcium and phosphate levels; CPK levels; urine myoglobin; arterial blood gas (ABG); PT, aPTT, and INR; and serum and urine toxic screening. Additionally, a medical professional may opt to employ CT scans and MRI. Skilled medical practitioners are familiar with the available tests and diagnostic tools that may be used to diagnose (or rule out) NMS.

If a patient develops NMS while on zonisamide therapy, the treating physician should search for other causes of NMS based on the criteria discussed above and known to medical professionals. Should no other obvious causes be identified, zonisamide may be removed, reduced, or alternatively tapered off to an acceptable level, and alternative treatment for the underlying medical condition be initiated as clinically indicated. If another cause for the NMS is identified, then it may be possible to carefully rechallenge with zonisamide once the symptoms have subsided. If the patient again appears to be developing NMS or is diagnosed with NMS, then switching to another AED may be warranted.

In some cases, it may be possible to reduce the level of zonisamide to avoid NMS or other side-effects, while maintaining the therapeutic efficacy of the drug therapy. Such decisions may be made by an attending medical personnel, for example, after considering the severity of the NMS or other side effects in relation to the patient's need for continued zonisamide therapy.

Conventional support measures for mild or severe NMS are known to skilled medical professionals. Normally, the condition and its symptoms are transient and can be reversed once the cause has been identified and addressed. Also, complications from the treatment of the NMS or its symptoms can be addressed as they arise. For example, abruptly removing anti-epileptic drug therapy from an epileptic patient may result in more severe or more frequent seizures or status epilepticus. Therefore, removal of zonisamide therapy carries the risk of more severe seizures. However, a hospital physician or emergency medical personnel will have access to other pharmacological interventions for short-term control of generalized seizure activity such as either intravenous lorazepam, at a dose of 0.1 mg/kg, or diazepam at 0.2 mg/kg. If sedatives prove insufficient, then a patient also may be administered fosphenytoin, or in status epilepticus, phenobarbital, with careful monitoring for respiratory depression. Intravenous administration is preferred since this route will provide the most rapid attainment of therapeutic serum levels. Additionally, at the treating physician's discretion, an alternate AED may be substituted for zonisamide.

Prevalence In Zonisamide Treated Patients:

The pharmacovigilance data that were collected, reviewed and analyzed provided the following information in respect of the incidence of NMS. To date, a total of 3 cases (two adult and one pediatric) fulfill the criteria of potential neuroleptic malignant syndrome. These cases were reviewed in detail for evaluation of possible safety signals. They were all deemed serious, and in all cases, the patients recovered with appropriate treatment.

Cases ZONI000813 and ZONI000929 are adult cases from Japan. In both cases the patients carried a diagnosis of Parkinson's disease (PD) and were enrolled in a double blind, placebo controlled phase 11 study of zonisamide as a potential treatment for PD (Protocol AD810N-202-1). In both cases the development of NMS appeared after the withdrawal of zonisamide (from 2 days to 14 days, respectively). In the former case, the study blind was broken, and the patient was documented to be on study medication (zonisamide), while in the second case, the blind was not broken. Case ZONI000926 is a spontaneous pediatric case from the US. In all of these cases there were weak or no confounding factors present.

Estimates of exposure, based upon retail and mail order prescriptions, indicate that the number of unique patients taking zonisamide capsules in the U.S. is about 37,276 (total prescriptions per year/average number of prescriptions per patient per year less a calculated percentage decrease based on estimated annual dropouts) in the time between approval in 2000 and December 2002 . Hospital patient data for that period, however, is not available and is not reflected in the estimates. Estimates of patient exposure for Japan indicate that the number of unique patients taking zonisamide is about 1,185,177 for time beginning with the approval in Japan through December 2002. Japanese data includes prescription and hospital patient data. Exposure from clinical trials are not included in the U.S. or Japanese exposure estimates. Based on these statistics, the estimated number of patients exposed to zonisamide in the U.S. and Japan is 1,222,453 unique patients. This is a rather conservative estimate, assuring that the number of patients actually exposed to zonisamide is unlikely to be higher than the estimate provided. Similarly, the incidences of NMS estimated herein are unlikely to be higher than calculated. Based on the exposure data and number of NMS cases, the incidence of NMS in the U.S. and Japan is calculated to be about 1:37,276 unique patients and 1:592,588 unique patients, respectively.

The following examples are provided to support the practice of the present invention and are not meant and should not be construed to limit the scope of the claims appended hereto.

### EXAMPLE 1

A 65-year old male carried a diagnosis of Parkinson's Disease (PD). The patient was enrolled in a double-blind, placebo controlled Phase II study designed to assess the potential utility of zonisamide in the treatment of PD. The patient's symptoms of NMS appeared 2 days after cessation of zonisamide therapy. Patients with PD are known to be at a rare, but increased risk for NMS. But NMS can occur spontaneously. See Ueda, et al., Neurology, Vol. 52, pp. 777-781, 1999. The temporal relationship between the appearance of NMS following zonisamide therapy and the absence of strong confounding factors suggests a causal relationship between zonisamide and the development of NMS.

### EXAMPLE 2

A 53-year old male carried a diagnosis of Parkinson's Disease (PD). The patient was enrolled in a double-blind, placebo controlled Phase II study designed to assess the potential utility of zonisamide in the treatment of PD. The patient's symptoms of NMS appeared 14 days after cessation of study medication. Although the blind of the study was not broken, it appears from the case report that the patient had been receiving zonisamide. The temporal relationship between the appearance of NMS following zonisamide therapy and the absence of strong confounding factors suggests a causal relationship between zonisamide and the development of NMS.

### EXAMPLE 3

A 5-year old male carried a diagnosis of cerebral palsy, quadriplegia, and epilepsy, and had recently been hospitalized for a tonsillectomy and adenoidectomy. Symptoms of NMS developed 3 weeks following the surgery. It was unclear whether the patient had been adequately hydrated prior to developing NMS. The patient had no known risk factors for developing NMS, except possible dehydration and restraint. See Sachev, et al., Am. J. Psychiatry, Vol. 154, pp. 1156-1158, 1997; and Ayad, Perioperative Medicine and Pain, Vol. 22, pp. 134-142, 2003. The presence of only weak confounding factors implicates a possible causal relationship between zonisamide and the development of NMS.

While this invention has been described with respect to various specific examples and embodiments, it is to be understood that the invention is not limited thereby and should only be construed by interpretation of the scope of the appended claims.

1. A method of using zonisamide as an adjunctive therapy for partial seizures to improve the safety of such therapy comprising:
providing a patient with a therapeutically effective amount of zonisamide, and
informing the patient or the patient's guardian during the course of zonisamide therapy that dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis are symptoms of NMS that require prompt medical evaluation if such symptoms are experienced by the patient.

2. The method of claim 1, wherein the therapeutically effective amount of zonisamide is from 25 mg to 600 mg.

3. The method of claim 1, wherein the therapeutically effective amount of zonisamide is provided in unit dose form.

4. The method of claim 1, wherein the therapeutically effective amount of zonisamide is provided in a unit dose form and in multiple doses to provide for a course of therapy.

5. The method of claim 4, wherein the unit dose is from 25 mg to 200 mg.

6. A method of using zonisamide as an adjunctive therapy for partial seizures to improve the health of a patient receiving such therapy comprising:
providing a patient with a therapeutically effective amount of zonisamide, and
informing the patient or the patient's guardian during the course of such therapy that dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis are symptoms of NMS that require prompt medical evaluation if such symptoms are experienced by the patient.

7. The method of claim 6, wherein the therapeutically effective amount of zonisamide is from 25 mg to 600 mg.

8. The method of claim 7, wherein the therapeutically effective amount of zonisamide is provided in unit dose form.

9. The method of claim 6, wherein the therapeutically effective amount of zonisamide is provided in a unit dose form and in multiple doses to provide for a course of therapy.

10. The method of claim 9, wherein the unit dose is from 25 mg to 200 mg.

11. A method of using zonisamide as an adjunctive therapy for partial seizures to reduce the risk of NMS in a patient receiving such therapy comprising:
providing the patient with a therapeutically effective amount of zonisamide, and
informing the patient or the patient's guardian during the course of zonisamide therapy that dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis are symptoms of NMS that require prompt medical evaluation if such symptoms are experienced by the patient.

12. The method of claim 11, wherein the therapeutically effective amount of zonisamide is from 25 mg to 600 mg.

13. The method of claim 12, wherein the therapeutically effective amount of zonisamide is provided in unit dose form.

14. The method of claim 11, wherein the therapeutically effective amount of zonisamide is provided in a unit dose form and in multiple doses to provide for a course of therapy.

15. The method of claim 14, wherein the unit dose is from 25 mg to 200 mg.

16. A method of using zonisamide as an adjunctive therapy for partial seizures comprising:
enhancing the safety profile of zonisamide by informing a prescribing physician that NMS may result from zonisamide therapy and to monitor a patient who is prescribed zonisamide as an adjunctive therapy for dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis;
recommending that, when dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis is observed, an appropriate diagnostic be employed by the physician to determine whether NMS is present; and
recommending that the physician remove, reduce, or taper off zonisamide dosing in the patient and initiate appropriate supportive therapy.

17. The method of claim 16, wherein the diagnostic comprises liver function tests (LFTs).

18. The method of claim 16, wherein the diagnostic comprises a complete blood count (CBC).

19. A method of using zonisamide as an adjunctive therapy for partial seizures comprising:
improving patient outcome by informing an emergency medical worker that a patient who is receiving zonisamide as an adjunctive therapy for partial seizures and exhibits dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis may be suffering from NMS; and
recommending performance of an appropriate diagnostic to determine whether NMS is present,
and if NMS is present, recommending that the worker initiate appropriate supportive therapy and discontinue zonisamide dosing in the patient.

20. The method of claim 19, wherein the diagnostic comprises liver function tests (LFTs).

21. The method of claim 19, wherein the diagnostic comprises a complete blood count (CBC).

22. A method of using zonisamide as an adjunctive therapy for partial seizures comprising:
providing packaging that includes a pharmaceutical formulation of zonisamide along with information providing a warning that zonisamide may cause NMS in some patients and that one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis are potentially signs of NMS; and
providing the packaging to a patient who has been prescribed zonisamide.

23. A method of using zonisamide as an adjunctive therapy for partial seizures comprising:
enhancing the safety of zonisamide by packaging a pharmaceutical formulation of zonisamide along with information providing a warning that zonisamide may cause NMS in some patients and that one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis are potentially signs of NMS and providing such packaging to a patient who has been prescribed zonisamide therapy.

24. A method of using zonisamide as an adjunctive therapy for partial seizures comprising:
administering a therapeutically effective amount of zonisamide to a subject in need of treatment;
observing the subject for the appearance of at least one symptom of NMS; and
if at least one symptom of NMS is observed, reducing the dosage of the zonisamide to a dosage that does not produce the at least one symptom of NMS.

25. The method of claim 24, wherein if at least one symptom of NMS is observed, administration of zonisamide is ceased.

26. The method of claim 24, wherein if at least one symptom of NMS is observed, the patient is tested for NMS.

27. The method of claim 26, wherein the testing comprises at least one measurement of complete blood count (CBC) or a liver function tests (LFTs).

28. The method of claim 25, further comprising administering a therapeutically effective amount of zonisamide after at least one symptom of NMS has subsided.

29. The method of claim 24, wherein the therapeutically effective amount of zonisamide is from 25 mg to 600 mg.

30. The method of claim 25, wherein the therapeutically effective amount of zonisamide is provided in unit dose form.

31. The method of claim 30, wherein the therapeutically effective amount of zonisamide is provided in a unit dose form and in multiple doses to provide for a course of therapy.

29. A method of administering zonisamide as an adjunctive therapy for partial seizures comprising:
providing a patient with a therapeutically effective amount of zonisamide and a therapeutically effective amount of at least one other anti-epilepsy drug; and
informing the patient or the patient's guardian that dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis are potentially signs of NMS that require prompt medical evaluation if such symptoms are experienced by the patient.

30. The method of claim 29, wherein the patient is informed by reference to a package drug insert.

31. The method of claim 30, wherein the patient's guardian is informed by reference to a package drug insert.

32. A method of using zonisamide as an adjunctive therapy for partial seizures comprising:
advising a physician prescribing zonisamide to a patient to monitor the patient for one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis,
recommending that when dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis is observed, an appropriate diagnostic be employed by the physician to determine whether NMS is present; and
recommending that the physician remove, reduce, or taper off zonisamide dosing in the patient and initiate appropriate supportive therapy.

33. A method for using zonisamide as an adjunctive therapy for partial seizures prescribed by a physician comprising:
monitoring a patient who is receiving administrations of zonisamide for one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis;
if one or more of said symptoms are observed, determining whether NMS is present in the patient; and
if NMS is diagnosed, reducing the zonisamide dosing until the patient's symptoms have subsided.

34. The method of claim 33, wherein the zonisamide dosing is increased after the patient's symptoms have subsided.

35. A method of using zonisamide as an adjunctive therapy for partial seizures prescribed by a physician comprising:
monitoring a patient who is receiving administrations of zonisamide for one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis;
if one or more of said symptoms are observed, determining whether NMS is present in the patient; and
if NMS is diagnosed, ceasing the zonisamide dosing until the symptoms of NMS have subsided.

36. The method of claim 35, wherein the zonisamide dosing is restored after the patient's symptoms have subsided.

37. A method of using zonisamide as an adjunctive therapy for partial seizures comprising:
providing packaging that includes a pharmaceutical formulation of zonisamide along with information providing a warning that zonisamide may cause NMS in patients who are restrained, dehydrated, or have Parkinson's disease; and that one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; and metabolic acidosis are potentially signs of NMS; and
providing the packaging to a patient who has been prescribed zonisamide.

38. A method of using zonisamide as an adjunctive therapy for partial seizures comprising:
enhancing the safety of zonisamide by packaging a pharmaceutical formulation of zonisamide along with information providing a warning that zonisamide may cause NMS in patients that are restrained, dehydrated, or have Parkinson's disease; and that one or more symptoms chosen from the group of dehydration; hyperthermia; muscular rigidity; altered mental status; tachycardia; dysphagia; hypertension or hypotension; diaphoresis or sialorrhea; tremor; incontinence; increased creatine phosphokinase (CPK) or urinary myoglobin; leukocytosis; shuffling gait; or metabolic acidosis are potentially signs of NMS and providing such packaging to a patient who has been prescribed zonisamide therapy.
